(19) United States
(12) Patent Application Publication
(10) Pub. No.: US 2014/0214065 A1
(43) Pub. Date: Jul. 31, 2014

(54) BLOOD LANCET DEVICE

(71) Applicant: Yong Pil KIM, Gimpo-si (KR)

(72) Inventor: Yong Pil KIM, Gimpo-si (KR)

(21) Appl. No.: 13/785,478

(22) Filed: Mar. 5, 2013

(30) Foreign Application Priority Data
Jan. 29, 2013 (KR) 10-2013-0009970

(51) Int. Cl. A61B 17/34

(52) U.S. Cl. CPC A61B 17/3417; A61B 17/3494
USPC 606/172; 606/182

(57) ABSTRACT

Disclosed is a blood lancet device including a shaft which is driven toward a lancet, a blood collection button for launching the shaft, a tensioning unit for tensioning the shaft, and a depth-adjusting unit for adjusting a puncturing depth of a needle of the lancet. The shaft is provided inside a body, tensioned by operation of the tensioning unit, and combined with the lancet at a front end thereof. The blood collection button is provided outside the shaft, elastically supported by an inside surface of the body, and arranged to protrude outward from an end of the body. The tensioning unit is arranged in line with the blood collection button. The depth-adjusting unit is combined with a front end of the body in a rotatable manner, has a hole provided in a center portion of a front surface thereof, and covers the lancet.

### CROSS-REFERENCES TO RELATED APPLICATION

This patent application claims the benefit of priority from Korean Patent Application No. 10-2013-0009970, filed on Jan. 29, 2013, the contents of which are incorporated herein by reference.

### BACKGROUND OF THE INVENTION

1. Field of the Invention

The present invention relates to a blood lancet device and, more particularly, to a blood lancet device used to take a blood sample for a medical checkup or disease diagnosis.

2. Description of the Related Art

Blood lancet devices are generally used by diabetics, who have to self-administer periodic blood sugar tests, and also by health care professionals in various institutes where blood drawing or blood collection is necessary and frequently carried out.

A conventional blood lancet device is structured such that a lancet made up of a fixed needle is inserted into a patient's body, and an elastic force is applied to the lancet so that the needle will drive into the skin of the patient and reach a predetermined depth in order to draw a small amount of blood to be used as a sample. For example, U.S. Pat. No. 4,517,978 discloses a technology in which a shaft combined with the lancet is pulled back and, when a blood collection button is pressed down, the shaft is driven forward so that the needle of the lancet is inserted rapidly into the skin and then retracted.

As for the structure of the above-described blood lancet device, the blood collection button for launching the lancet protrudes from an upper end of the body, and a lever which is used to tension the lancet is located at a rear end of the body. Because of this structure, when a user pulls the shaft back to tension the lancet for the purpose of collecting a blood sample, the user of the blood lancet device needs to hold the body of the blood lancet device with one hand and, in such a state, pull the shaft back and then push the blood collection button with the other hand. This is a bothersome and inconvenient operation because the user needs to use both hands.

In addition, the structure in which the lever used to pull the shaft back is located at the rear end of the body leads to an increased length of the blood lancet device and a complicated structure, which are obstacles in making the blood lancet device compact. For this reason, although blood sugar testers are decreasing in size as the result of the development of technologies, and improved functions which increase the efficiency of the devices, miniaturization of the blood lancet device, which is one of the essential parts or accessories of the blood sugar tester, has not been successful. This causes an inconvenience when packaging the blood sugar tester in single units.

The foregoing is intended merely to aid in the understanding of the background of the present invention, and is not intended to mean that the present invention falls within the purview of the related art that is already known to those skilled in the art.

[Documents of Related Art]

(Patent Document 1) U.S. Pat. No. 4,517,978 (Issued on May 21, 1985)

(Patent Document 2) Korean Patent Registration No. 10-1208479 (Publication Date: Dec. 5, 2012)

### SUMMARY OF THE INVENTION

Accordingly, the present invention has been made keeping in mind the above problems occurring in the related art, and the present invention is intended to propose a blood lancet device having advantages of eliminating the inconvenience of a user needing to use both hands when tensioning and launching a lancet by arranging a tensioning lever used to tension the lancet to be in line with a blood collection button used to launch the lancet, reducing the total length of the whole blood lancet device by arranging the blood collection button and the tensioning lever to be in line with each other on one surface of the body, and allowing the blood lancet device to be packaged in single units.

In order to achieve the above object, according to one aspect of the present invention, there is provided a blood lancet device including: a shaft provided inside a body and tensioned by operation of a tensioning unit, a front end of the shaft being combined with a lancet; a blood collection button provided outside the shaft, elastically supported by an inside surface of the body, and arranged to protrude outward from an end of the body, the blood collection button that is used to launch the shaft; the tensioning unit arranged in line with the blood collection button, the tensioning unit being used to tension the shaft; and a depth-adjusting unit which is combined with a front end of the body in a rotatable manner, has a hole provided in a center portion of a front surface thereof, covers the lancet, and adjusts a puncturing length of a needle of the lancet according to rotation of the depth-adjusting unit.

The shaft may include: a stopping protrusion which is provided on a surface of the shaft corresponding to the blood collection button and controls a pressing-down operation of the blood collection button; a stopping member which moves the shaft according to a tensioning operation of the tensioning unit and which is provided on a surface of the shaft corresponding to the tensioning unit; and a step portion which protrudes outward from a rear end of the shaft, wherein the tensioning operation is performed in such a manner that when the tensioning unit is pulled back, the shaft is moved backward by the stopping member which is latched by the tensioning unit, and the stopping protrusion is latched by one side surface of the blood collection button by movement of the shaft.

The tensioning unit may include: a tensioning lever having an inside surface, a protruding piece which engages with the stopping member of the shaft and is formed on the inner surface, and an upper end portion which protrudes outward from a surface of the body, the tensioning lever being combined with an outside surface of the shaft in a slidable manner; and a tensioning spring which is elastically installed between the protruding piece of the tensioning lever and the step portion formed at the rear end portion of the shaft, the tensioning spring being used to return the tensioning lever which is moved backward for the purpose of tensioning the shaft to an original position.

The blood lancet device may further include a main spring which is elastically inserted between the rear end portion of the shaft and the body, the main spring being compressed when the shaft is tensioned and returning the tensioned shaft to an original position when the blood collection button is pressed down.

The blood collection button may include: a button guide which has a cylindrical body combined with the outer surface of the shaft, a helical groove formed on the outer surface of the cylindrical body, and a through hole formed behind the helical groove and extending in a direction perpendicular to a longitudinal direction of the shaft; and a button which is inserted in the through hole of the button guide, through a center of which the shaft penetrates, and which has a lower end portion made of an elastic piece so as to be elastically supported by an inside surface of the body, and an upper end portion protruding outward from the body.

The depth-adjusting unit may include: an inner ring provided with a protrusion which is formed in an inside surface thereof and is inserted in the helical groove of the button guide so that the inner ring is combined with a portion of the button guide which is the portion located outside the helical groove; an outer ring combined with an outside surface of the inner ring; and an adjusting cap which is combined with the outer ring in a removable manner and which has a hole at a center portion of a front surface thereof.

In the blood lancet device, a plurality of position-determining grooves may be formed at regular intervals along an outer peripheral surface of a leading end portion of the button guide, a position-determining protrusion which is to be selectively inserted into one of the position-determining grooves may be formed on the inside surface of the inner ring, a movement range of the inner ring which moves along the helical groove of the button guide may change stepwise by the selective insertion of the position-determining protrusion, and a length of a needle of the lancet which is exposed to the outside may be adjusted stepwise by the stepwise movement of the inner ring.

According to the blood lancet device of the present invention, since the tensioning lever which is used to tension the lancet is arranged to be in line with the blood collection button which is used to launch the lancet, a user can tension and launch the lancet with one hand, that is, the same hand which is holding the body of the blood lancet device. Therefore, the inconvenience of the user needing to use both hands for tensioning and launching the lancet is eliminated.

In addition, since the blood collection button and the tensioning lever are provided in line with each other on one surface of the body of the blood lancet device, the length of the whole blood lancet device is decreased. For this reason, the blood lancet device can be made compact, and can be packaged in single units.

Furthermore, since the puncturing depth of the needle of the lancet can be adjusted depending on the user, the pain which is inflicted on the user is decreased. Still further, the used lancet can be easily replaced with a new one.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a perspective view illustrating a blood lancet device according to one embodiment of the present invention;

FIG. 2 is a side elevation view illustrating the blood lancet device according to the embodiment of the present invention;

FIG. 3 is a cross-sectional perspective view illustrating the blood lancet device according to the embodiment of the present invention;

FIG. 4 is an exploded side elevation view illustrating a depth-adjusting unit of the blood lancet device according to the embodiment of the present invention;

FIG. 5 is a cross-sectional view illustrating a state in which a position-determining protrusion of an inner ring is selectively inserted in one of position-determining grooves of a button guide depending on the degree of rotation of the inner ring of the depth-adjusting unit of FIG. 4;

FIG. 6 is a cross-sectional view illustrating a state in which a shaft is pushed back by a tensioning lever of the blood lancet device according to the embodiment of the present invention; and

FIG. 7 is a cross-sectional view illustrating a state in which the tensioning lever is driven as a blood collection button of the blood lancet device according to the embodiment of the present invention is pushed.

### DETAILED DESCRIPTION OF THE INVENTION

The above and other objects, features and other advantages of the present invention will be more clearly understood from the following detailed description when taken in conjunction with the accompanying drawings. Hereinbelow, a blood lancet device of the preferred embodiment of the present invention will be described in detail with reference to the accompanying drawings. The identical reference numerals denote substantially identical or equivalent components otherwise described differently.

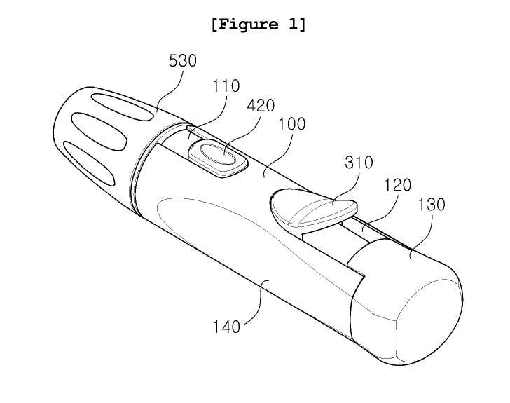

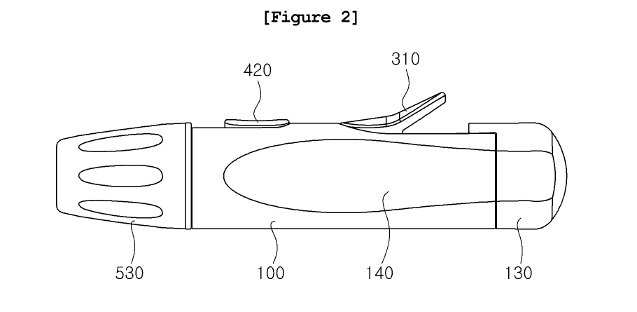

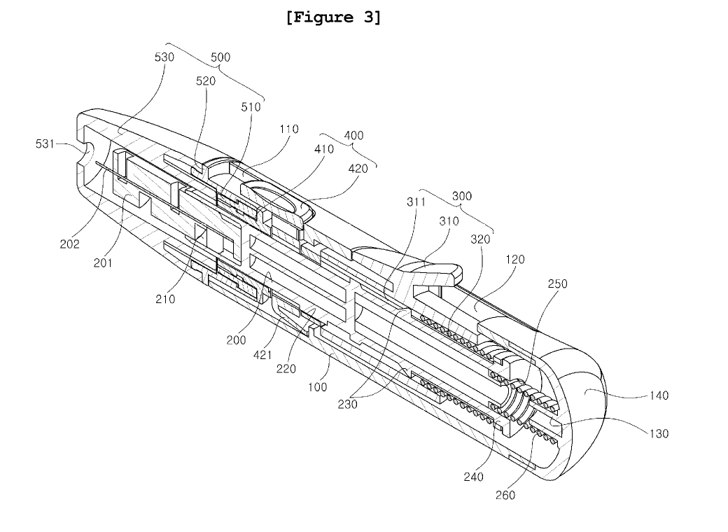

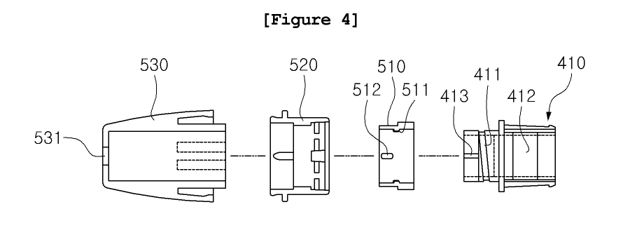

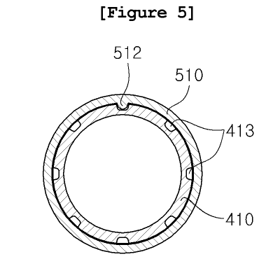

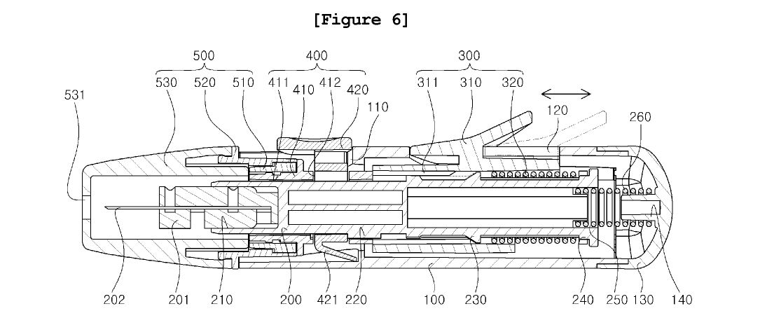

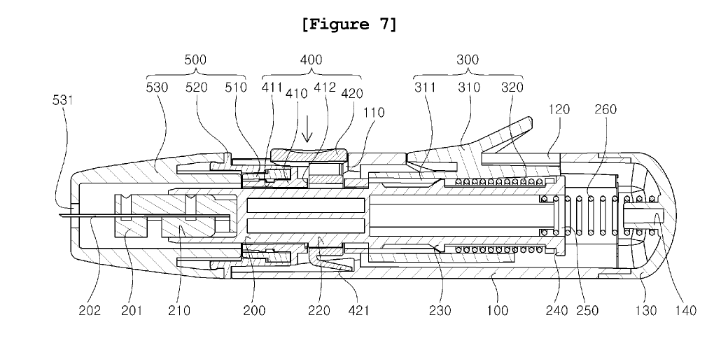

FIG. 1 is a perspective view illustrating a blood lancet device according to one embodiment of the present invention, FIG. 2 is a side elevation view illustrating the blood lancet device according to the embodiment of the present invention, FIG. 3 is a cross-sectional perspective view illustrating the blood lancet device according to the embodiment of the present invention, FIG. 4 is an exploded cross-sectional view illustrating a depth-adjusting unit of the blood lancet device according to the embodiment of the present invention, FIG. 5 is a cross-sectional view illustrating a state in which a position-determining protrusion of an inner ring is selectively inserted into one of position-determining grooves of a button guide, FIG. 6 is a cross-sectional view illustrating a state in which a shaft is tensioned by a tensioning lever in the blood lancet device according to the present invention, and FIG. 7 is a sectional view illustrating a state in which a blood collection button of the blood lancet device is pressed down so that the tensioned shaft is driven forward.

A blood lance device according to the present invention, illustrated in FIGS. 1 to 3, includes a body 100, a shaft 200 which is provided in the body 100 and has a front end to which a lancet 201 is connected, a tensioning unit 300 which is used to tension the shaft 200, a blood collection button 400 which is used to drive the tensioned shaft 200 forward, and a depth-adjusting unit 500 which adjusts a puncturing depth of a needle 202 of the lancet.

The body 100 is formed of a hollow cylinder. The body 100 has a first through hole 110 on an upper surface thereof, and the blood collection button 400 is installed in the first through hole 110. The body 100 has a second through hole 120 which is in line with and distanced from the first through hole 110. The tensioning unit 300 is installed in the second through hole 120. A rear cap 130 which can be removed from the body 100 is provided at a rear end of the body 100 so that parts to be provided in the body 100 can be easily assembled through the rear end due to the removable rear cap 130. The outside surface of the body 100 includes anti-slipping surfaces 140 with which a user's fingers or hand come in contact. This offers comfort and an anti-slipping function, thereby improving a user's convenience.

A leading end of the shaft 200 is provided with a combination hole 210 to which the lancet 201 is connected. The shaft 200 is provided with a stopping protrusion 220 in an outside surface thereof, at a location corresponding to the blood collection button 400 installed in the first through hole 110. The shaft 200 is further provided with a stopping member 230 in the outside surface thereof, at a location corresponding to the tensioning unit 300 installed in the second through hole 120. The stopping member 230 may be plural in number, and the plurality of stopping members 230 may be arranged at regular intervals on the surface of the shaft 200. A step portion 240 is provided in a manner to protrude from a rear end of the shaft 200. An accommodation portion 250 is formed inside a rear end portion of the shaft 200 to accommodate and support a main spring 260. The main spring 260 is interposed between the accommodation portion 250 of the shaft 200 and the rear end cap 130 so that the shaft 200 will elastically move. A fixing step 131 to fix an end of the main spring 260 is formed inside the rear end cap 130, in a protruding manner, which corresponds to the accommodation portion 250.

The tensioning unit 300 is provided at a location facing the stopping member 230 of the shaft 200 in a manner to be able to slide along the outside surface of the shaft 200. The tensioning unit 300 is made up of a tensioning lever 310 which protrudes from an end of the body 100, while passing through the second through hole 120 formed in the upper surface of the body 100, and a tensioning spring 320 which elastically supports the tensioning lever 310. The tensioning lever 310 includes multiple protruding pieces 311 which will engage with the stopping members 230 which are formed at regular intervals on the outside surface of the shaft 200. The protruding pieces 311 are formed on an inside surface of the tensioning lever 310. The tensioning spring 320 is elastically interposed between the protruding pieces 311 of the tensioning lever 310 and the step portion 240 of the shaft 200.

The blood collection button 400 includes a button guide 410 and a button 420. The button guide 410 is formed of a cylindrical body combined with the outside surface of the shaft 200. The cylindrical body has a helical groove 411 on the outside surface. The button guide 410 also has a through hole 412 which is formed behind the helical groove 411 and extends in a direction perpendicular to the longitudinal direction of the shaft 200. The button 420 includes a cylindrical body which is inserted in the through hole 412 of the button guide 410. The shaft 200 passes through the center of the cylindrical body. An elastic piece 421 branches from a lower end of the cylindrical body and is elastically supported in the body 100. The button 420 is elastically supported by the inside surface of the body 100, and an upper portion of the button 420 protrudes outward from the body 100 through the first through hole 110 of the body 100.

The button guide 410 and the button 420 are installed on the outside surface of the shaft 200 at the location corresponding to the first through hole 110 of the body 100 in such a manner that the shaft 200 can move. In this case, when the stopping protrusion 220 of the shaft 200 is located inside the button 420, the button 420 is pressed down by the stopping protrusion 220 and is thus lowered. On the other hand, when the stopping protrusion 220 of the shaft 200 is outside the button 420 and is located inside the button guide 410, the button 420 is lifted to a predetermined height by the elastic force of the elastic piece 421 and the stopping protrusion 220 located inside the button guide 410 is stopped by the side surface of the button 420 which is lifted to the predetermined height.

As illustrated in FIGS. 3 and 4, the depth-adjusting unit 500 includes an inner ring 510. A protrusion 511 to be inserted into the helical groove 411 of the button guide 410 is formed in an inside surface of the inner ring 510. The inner ring 510 is combined with a portion of the button guide 410 which is located outside the helical groove 411. The depth-adjusting unit 500 further includes an outer ring 520 combined with an outside surface of the inner ring 510, and an adjusting cap 530 which is combined with the outer ring 520 in a removable manner, has a hole 531 at the center of a front surface, and covers the lancet 201.

Next, operation of the depth-adjusting unit 500 is described. When a user holds and rotates the adjusting cap 530, the inner ring 510 is rotated together with the outer ring 520 which connects the adjusting cap 530 to the inner ring 510. This rotation is performed such that the protrusion 511 of the inner ring which is combined with the helical groove 411 of the button guide 410 moves forward or backward along the helical groove 411 according to the direction of rotation. That is, the length of a needle 202 of the lancet 201 which protrudes from the hole 520 of the front surface of the depth-adjusting cap 520 and is exposed to the outside changes depending on the forward movement or backward movement of the depth-adjusting unit 500.

In addition, as illustrated in FIG. 5, a plurality of position-determining grooves 413 is formed at regular intervals along an outside circumferential surface of a leading end portion of the button guide 410 in which the helical groove 411 is formed.

A position-determining protrusion 512 to be inserted into one of the position-determining grooves 413 is formed in the inside surface of the inner ring 510. One of the position-determining grooves 413 into which the position determining protrusion 512 is inserted is determined according to the distance by which the inner ring 510 moves along the helical groove 411 of the button guide 410. Depending on into which position-determining groove 413 the position-determining protrusion 512 is inserted, the moving distance is adjusted stepwise in the manner of a first, second, third, and fourth stage. According to the stage, the length of the needle 202 of the lancet which is exposed is adjusted stepwise and the puncturing depth of the needle 202 of the lancet is also adjusted stepwise.

Hereinafter, operation of the blood lancet device having the structure described above will be described.

First, a user adjusts the puncturing depth of the needle 202 of the lancet according to user's desire by rotating the depth-adjusting unit 500. That is, the user adjusts the length of the needle 202 of the lancet which protrudes from the hole of the adjusting cap 530 and is exposed to the outside by rotating the adjusting cap 530 stepwise.

As illustrated in FIG. 6, when the tensioning lever 310 provided on the rear side of the button 420 is pulled back, the protruding piece 311 formed on the inside surface of the tensioning lever 310 pushes the stopping member 230 formed on the outside surface of the shaft 200 so that the shaft 200 is moved backward. As the shaft 200 is moved backward, the stopping protrusion 220 of the shaft 200 moves from the inside of the button 420 and to the inside of the button guide 410 so that pressing force applied to the button 420 is released. The button 420 to which the pressing force is not applied any more is lifted to a predetermined height due to the elastic force of the elastic piece 421, the stopping protrusion 220 positioned inside the button guide 410 is stopped by the side surface of the button 420 which is lifted to the predetermined height, and as a result, the shaft 200 is tensioned. At this time, the main spring 260 is compressed by the tensioning of the shaft 200 and the tensioning lever 310 is returned back to the original position by the elastic force of the tensioned spring 320 at the same time as the shaft 200 is tensioned.

When the button 420 is pressed down while the front end of the adjusting cap 530 is in contact with the skin in the state illustrated in FIG. 6, the button moves downward 420 and the stopping protrusion 220 which is latched by the side surface of the button 420 is released. At this time, the main spring 260 is recovered to its original form, the shaft 200 moves forward, and the needle 202 of the lancet 201 combined with the shaft 200 is shot into the skin.

After the lancet 201 is used once, the adjusting cap 530 which is removable is removed from the outer ring 520. Then, the used lancet 201 is extracted and removed through the combination hole 210 of the shaft, a new lancet 210 is inserted into the combination hole 210 of the shaft 200, and the adjusting cap 530 is assembled with the outer ring 520. In this way, the used lancet can be replaced by a new lancet.

As described above, since the tensioning lever which is used to tension the lancet is arranged in line with the blood collection button which is used to launch the lancet, a user can tension and launch the lancet with one hand, that is, the same hand which is holding the body of the blood lancet device. Therefore, the inconvenience of the user needing to use both hands for tensioning and launching the lancet is eliminated.

In addition, since the blood collection button and the tensioning lever are provided in line with each other on one surface of the body of the blood lancet device, the length of the whole blood lancet device is decreased. For this reason, the blood lancet device can be made compact, and can be packaged in single units.

Furthermore, since the puncturing depth of the needle of the lancet can be adjusted depending on the user, the pain which is inflicted on the user is decreased. Still further, the used lancet can be easily replaced with a new one.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A blood lancet device comprising:
a shaft provided inside a body and tensioned by operation of a tensioning unit, a front end of the shaft being combined with a lancet;
a blood collection button provided outside the shaft, elastically supported by an inside surface of the body, and arranged to protrude outward from an end of the body, the blood collection button being used to launch the shaft;
the tensioning unit arranged in line with the blood collection button, the tensioning unit being used to tension the shaft; and
a depth-adjusting unit which is combined with a front end of the body in a rotatable manner, has a hole provided in a center portion of a front surface thereof, covers the lancet, and adjusts a puncturing length of a needle of the lancet according to rotation of the depth-adjusting unit.

2. The blood lancet device according to claim 1, wherein the shaft includes:
a stopping protrusion which is provided on a surface of the shaft corresponding to the blood collection button and controls a pressing-down operation of the blood collection button;
a stopping member which moves the shaft according to a tensioning operation of the tensioning unit and which is provided on a surface of the shaft corresponding to the tensioning unit; and
a step portion which protrudes outward from a rear end of the shaft, and
wherein the tensioning operation is performed in such a manner that when the tensioning unit is pulled back, the shaft is moved backward by the stopping member which is latched by the tensioning unit, and the stopping protrusion is latched by one side surface of the blood collection button by movement of the shaft.

3. The blood lancet device according to claim 2, wherein the tensioning unit includes:
a tensioning lever having an inside surface, a protruding piece which engages with the stopping member of the shaft and is formed on the inner surface, and an upper end portion which protrudes outward from a surface of the body, the tensioning lever being combined with an outside surface of the shaft in a slidable manner; and
a tensioning spring which is elastically installed between the protruding piece of the tensioning lever and the step portion formed at the rear end portion of the shaft, the tensioning spring being used to return the tensioning lever which is moved backward for the purpose of tensioning the shaft to an original position.

4. The blood lancet device according to claim 1, further comprising a main spring which is elastically inserted between the rear end portion of the shaft and the body, the main spring being compressed when the shaft is tensioned, and returning the tensioned shaft to an original position when the blood collection button is pressed down.

5. The blood lancet device according to claim 1, wherein the blood collection button includes:
a button guide which has a cylindrical body combined with the outer surface of the shaft, a helical groove formed on the outer surface of the cylindrical body, and a through hole formed behind the helical groove and extending in a direction perpendicular to a longitudinal direction of the shaft; and
a button which is inserted in the through hole of the button guide, through a center of which the shaft penetrates, and which has a lower end portion made of an elastic piece so as to be elastically supported by an inside surface of the body, and an upper end portion protruding outward from the body.

6. The blood lancet device according to claim 5, wherein the depth-adjusting unit includes:
an inner ring provided with a protrusion which is formed in an inside surface thereof and is inserted in the helical groove of the button guide so that the inner ring is combined with a portion of the button guide which is the portion located outside the helical groove;
an outer ring combined with an outside surface of the inner ring; and
an adjusting cap which is combined with the outer ring in a removable manner and which has a hole at a center portion of a front surface thereof.

7. The blood lancet device according to claim 6, wherein a plurality of position-determining grooves is formed at regular intervals along an outer peripheral surface of a leading end portion of the button guide, a position-determining protrusion which is to be selectively inserted into one of the position-determining grooves is formed on the inside surface of the inner ring, a movement range of the inner ring which moves along the helical groove of the button guide changes stepwise by the selective insertion of the position-determining protrusion, and a length of a needle of the lancet which is exposed to outside is adjusted stepwise by the stepwise movement of the inner ring.
